(19) United States
(12) Patent Application Publication
(10) Pub. No.: US 2020/0372638 A1
(43) Pub. Date: Nov. 26, 2020

(54) AUTOMATED SCREENING OF HISTOPATHOLOGY TISSUE SAMPLES VIA CLASSIFIER PERFORMANCE METRICS

(71) Applicant: DECIPHEX, Dublin (IE)

(72) Inventors: Mark GREGSON, Dublin (IE); Donal O'SHEA, Dublin (IE)

(21) Appl. No.: 16/766,916

(22) PCT Filed: Nov. 27, 2018
(86) PCT No.: PCT/EP2018/082742

§ 371 Date: May 26, 2020

Related U.S. Application Data
(60) Provisional application No. 62/590,866, filed on Nov. 27, 2017.

(51) Int. Cl. G06T 7/00; G06T 7/11; G16H 10/40; G16H 50/20; G16H 50/70; G16H 30/40; G06N 20/00

(52) U.S. Cl. CPC G06T 7/0012; G06T 7/11; G16H 10/40; G16H 50/20; G16H 50/70; G06T 2207/30024; G06N 20/00; G06T 2207/10056; G06T 2207/20081; G06T 2207/20084; G16H 30/40

(57) ABSTRACT

Systems and methods are provided for screening a set of histopathology tissue samples representing a region of interest for abnormalities. A pattern recognition classifier is trained on a first set of images, each representing a tissue sample that is substantially free of abnormalities, and a second set of images, each representing one of the set of histopathology tissue samples representing the region of interest. At least one performance metric from the pattern recognition classifier is generated. A given performance metric represents one of an accuracy of the classifier in discriminating between images representing tissue that is substantially free of abnormalities and images of histopathology tissue samples representing the region of interest and a training rate of the pattern recognition classifier. A likelihood of abnormalities in the region of interest is determined from the at least one performance metric from the pattern recognition classifier.

### RELATED APPLICATIONS

The present application claims priority to U.S. Provisional Patent Application Ser. No. 62/590,866 filed Nov. 27, 2017 entitled AUTOMATED SCREENING OF HISTOPATHOLOGY TISSUE SAMPLES VIA CLASSIFIER PERFORMANCE METRICS under Attorney Docket Number DECI-027172 US PRO, the entire contents of which being incorporated herein by reference in its entirety for all purposes.

### TECHNICAL FIELD

The present invention relates generally to the field of medical diagnostics, and more particularly to automated screening of histopathology tissue samples via an analysis of classifier performance metrics.

### BACKGROUND OF THE INVENTION

Histopathology refers to the microscopic examination of tissue in order to study the manifestations of disease. Specifically, in clinical medicine, histopathology refers to the examination of a biopsy or surgical specimen by a pathologist, after the specimen has been processed and histological sections have been placed onto glass slides. The medical diagnosis from this examination is formulated as a pathology report describing any pathological changes in the tissue. Histopathology is used in the diagnosis of a number of disorders, including cancer, drug toxicity, infectious diseases, and infarctions.

### SUMMARY OF THE INVENTION

In one implementation, a system is provided for screening a set of histopathology tissue samples representing a region of interest for abnormalities. The system includes a processor and a non-transitory computer readable medium storing executable instructions. The instructions include a pattern recognition classifier trained on a first set of images, each representing a tissue sample that is substantially free of abnormalities, and a second set of images, each representing one of the tissue samples representing the region of interest. A classifier evaluation component generates at least one performance metric from the pattern recognition classifier. A given performance metric represents one of an accuracy of the classifier in discriminating between images that are substantially free of abnormalities and images representing the set of tissue samples representing the region of interest and a training rate of the pattern recognition classifier. An anomaly detection component determines a likelihood of abnormalities in the region of interest from the at least one performance metric from the pattern recognition classifier. A user interface provides the determined likelihood to a user at an associated output device.

In another implementation, a method is provided for screening a set of histopathology tissue samples representing a region of interest for abnormalities. A pattern recognition classifier is trained on a first set of images, each representing a tissue sample that is substantially free of abnormalities, and a second set of images, each representing one of the set of histopathology tissue samples representing the region of interest. at least one performance metric is generated from the pattern recognition classifier. A given performance metric represents one of an accuracy of the classifier in discriminating between images representing tissue that is substantially free of abnormalities and images of histopathology tissue samples representing the region of interest and a training rate of the pattern recognition classifier. A likelihood of abnormalities in the region of interest is determined from the at least one performance metric from the pattern recognition classifier.

In yet another implementation, a system is provided for screening a set of histopathology tissue samples representing a region of interest for abnormalities. The system includes a processor and a non-transitory computer readable medium storing executable instructions. The instructions include a pattern recognition classifier trained on a first set of images, each representing a tissue sample that is substantially free of abnormalities, and a second set of images, each representing one of the tissue samples representing the region of interest. A classifier evaluation component determines an accuracy of the classifier in discriminating between images that are substantially free of abnormalities and images representing the set of tissue samples representing the region of interest. An anomaly detection component determines a likelihood of abnormalities in the region of interest as a function of the determined accuracy of the classifier. A user interface provides the determined likelihood to a user at an associated output device.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a functional block diagram of a system for screening histopathology tissue samples via a normal model;

FIG. 2 illustrates an example of a system for screening histopathology tissue samples from a region of interest;

FIG. 3 illustrates one example of a method for screening a set of histopathology tissue samples representing a region of interest for abnormalities; and

FIG. 4 is a schematic block diagram illustrating an exemplary system of hardware components capable of implementing examples of the systems and methods disclosed herein.

### DETAILED DESCRIPTION

Systems and methods are provided for automated screening of histopathology tissue samples via classifier performance metrics. Specifically, a pattern recognition classifier is trained on normal training images, that is, images of tissues free of abnormalities, and test images representing a region of interest, such as an organ of a patent. If the test images are significantly different from the normal images, indicating that the region of interest may include abnormalities, the classifier will be able to readily differentiate between them after training. If the test images resemble the normal images, indicating that the region of interest is substantially free from abnormalities, the classifier will struggle to differentiate between them. As a result, the test samples can be prescreened for abnormality in an automated manner by evaluating the performance of the classifier, requiring intervention of a pathologist only when an abnormality is found to be present.

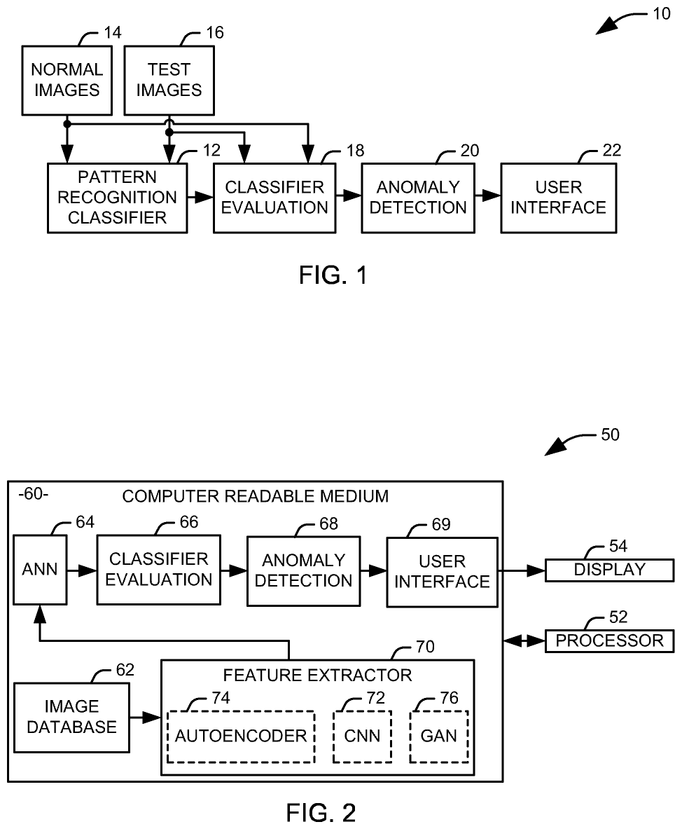

FIG. 1 illustrates a functional block diagram of a system 10 for screening histopathology tissue samples from a region of interest. It will be appreciated that the histopathology tissue samples can include tissue from the gastrointestinal system, the prostate, the skin, the breast, the kidneys, the liver, the lymph nodes, and any other appropriate location in a body of a human or animal subject. Tissue can be extracted via biopsy or acquired via excision or analysis of surgical specimens. In the case of animal subjects, tissue sections can be taken during an autopsy of the animal. The system 10 includes a classifier 12 trained on a plurality of normal images 14 and a plurality of test images 16 representing the region of interest. As described above, each of the plurality of normal images 14 represents a tissue sample that is substantially free of abnormalities, and each of the test images 16 represents unknown content. Accordingly, no images of specific tissue pathologies or other abnormalities is necessary for the training process. It will be appreciated that the normal images 14 and test images 16 can be acquired from stained histopathology tissue samples, which may have one or more stain normalization processes applied to standardize the images.

In practice, the images can be whole slide images, single frame capture images from a microscope mounted camera, or images taken during endoscopic procedures. The images can be brightfield, greyscale, colorimetric, or fluorescent images, and can be stored in any appropriate image format. Tissue abnormalities can include polyps, tumors, inflammation, infection sites, or other abnormal tissue within a body. In the liver, abnormalities can include infiltrate, glycogen, necrosis, vacuolation, hyperplasia, hypertrophy, fibrosis, hematopoiesis, granuloma congestion, pigment, arthritis, cholestasis, nodule, hemorrhage, and mitotic figures/regeneration. In the kidney, abnormalities can include infiltrate, necrosis, vacuolation, basophilic tubule, cast renal tubule, hyaline droplet, hyperplasia, fibrosis, hematopoiesis, degeneration/regeneration/mitotic figures, mineralization, dilation, hypoplasia, hypertrophy, pigment, nephropathy, glomerulosclerosis, cysts, congestion, and hemorrhage.

The classifier 12 can be implemented as any of a plurality of supervised learning algorithms, along with appropriate logic for extracting classification features from the normal images 14 and the test images 16. In one implementation, the extracted features can include both more traditional image processing features, such as color, texture, and gradients, as well as features derived from the latent space of a variational autoencoder. The training process of a given classifier will vary with its implementation, but the training generally involves a statistical aggregation of training data from a plurality of training images into one or more parameters associated with the output class.

Appropriate supervised learning algorithm for the classifier 12 can include, for support vector machines, self-organized maps, fuzzy logic systems, data fusion processes, ensemble methods, rule based systems, or artificial neural networks. For example, a support vector machine (SVM) classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class (e.g., “normal” or “test”) and an associated confidence value can be determined for a given input feature vector according to its position in feature space relative to the boundaries.

An artificial neural network classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. A variation of the artificial network is a convolution neural network, in which the hidden layers include one or more convolutional layers that learn a linear filter that can extract meaningful structure from an input image. Due to the ability of the convolution neural network to generate localized structure from regions of the image, it will be appreciated that, where a convolution neural network is utilized, the images 14 and 16 can be provided directly to the classifier 12 without additional feature extraction.

In another implementation, the classifier 12 can include a regression model configured to provide calculate a parameter representing a likelihood that a given image represents tissue with abnormalities. In practice, this value can be threshold to determine a final output class. A rule-based classifier applies a set of logical rules to the extracted features to select an output class. Generally, the rules are applied in order, with the logical result at each step influencing the analysis at later steps. In one implementation, multiple supervised learning algorithms can be used, with an arbitration element can be utilized to provide a coherent result from the plurality of classifiers.

A classifier evaluation component 18 generates at least one performance metric from the pattern recognition classifier 12. For example, the set of normal images 14 and the set of test images 16 can be divided into training sets, for training the classifiers, and validation sets, for testing the classifier performance. Accordingly, the trained classifier can be tested on a set of labeled validation images to determine a classifier accuracy in discriminating between images that are substantially free of abnormalities and images representing the set of tissue samples representing the region of interest, either after training or at a certain stage of training. A given performance metric can represent either an accuracy of the classifier or a training rate of the pattern recognition classifier, representing a number of training samples necessary to achieve a threshold level of accuracy.

An anomaly detection component 20 determines a likelihood of abnormalities in the region of interest from the at least one performance metric from the pattern recognition classifier. The likelihood can be determined, for example, as a function of the classifier accuracy after training, a function of a training rate of the classifier, or a function of both parameters. It will be appreciated that the likelihood is not necessarily a probability, with a value restricted between zero and one, but can also be a continuous variable ranging between different values or even a categorical value classifying the tissue as “normal” or “abnormal” or another set of suitable classes. A user interface 22 provides the calculated likelihood for the region of interest to a user at an associated output device (not shown), such as a display.

The determined likelihood can be used for triage, or prescreening, of tissue samples to be analyzed by pathologists, for research, or for diagnosis and monitoring of conditions in a patient. For example, a cohort of tissues can be ranked by the determined likelihood of abnormalities, allowing pathologists to triage and prioritize patient cases, with the most abnormal cased reviewed first. Alternatively, normal samples can be eliminated and an automatic report of negative findings can be provided, obviating the need for review by a pathologist. Normal samples, as used here, would have a likelihood of abnormality that is less than a set threshold, selected to provide the best performance of the system, based on specificity and sensitivity. The determined likelihood can also be used to identify abnormalities in the tissue and to evaluate therapeutic responses, predict outcomes, and evaluate biomarkers. In practice, the determined likelihood can be used to supplement the results of other classifiers applied to detect or identify abnormalities in the tissue.

FIG. 2 illustrates an example of a system 50 for screening histopathology tissue samples from a region of interest. The system 50 includes a processor 52 and a non-transitory computer readable medium 60 that stores executable instructions for evaluating histopathology tissue samples. In the illustrated implementation, the non-transitory computer readable medium 60 stores an image database 62 containing training and validation images for an artificial neural network (ANN) classifier 64. The image database contains both normal images, representing tissue samples that are substantially free of abnormalities, and test images representing the region of interest. The training images from the image database 62, representing both normal images and test images, can be provided to a feature extractor 70, which extracts classification features from the training images. It will be appreciated that, instead of storing the images in the image database 62, they could instead be provided directly to the feature extractor 70 from a remote system via a network interface (not shown).

The feature extractor 70 can process each image to provide a plurality of feature values for each image. In the illustrated implementation, this can include both global features of the image as well as regional or pixel-level features extracted from the image. In the illustrated implementation, the extracted features can include a first set of features generated from histograms of various image processing metrics for each of a plurality of regions, the metrics including values representing color, texture, and gradients within each region. Specifically, one set of features can be generated from multi-scale histograms of color and texture features. Another set of features can be generated via a dense Speeded-Up Robust Features (SURF) feature detection process.

Additional features can be generated from latent features generated by other expert systems. In the illustrated implementation, the features can include latent vectors generated by a convolutional neural network 72 (CNN), an autoencoder 74, such as a variational autoencoder, and a generative adversarial network (GAN) 76. It will be appreciated that each of the convolutional neural network 72, the autoencoder 74, and the generative adversarial network 76 are trained on the set of training images 62. The convolutional neural network 72, in general terms, is a neural network that has one or more convolutional layers within the hidden layers that learn a linear filter that can extract meaningful structure from an input image. As a result, one or more hidden layers of the convolutional neural network 72 can be utilized as classification features.

The autoencoder 74 is an unsupervised learning algorithm that applies backpropagation to an artificial neural network, with the target values to be equal to the inputs. By restricting the number and size of the hidden layers in the neural network, as well as penalizing neuron activation, the neural network defines a compressed, lower dimensional representation of the image in the form of latent variables, which can be applied as features for anomaly detection. In one implementation, the autoencoder 74 is a variational autoencoder, that works similarly, but restricts the distribution of the latent variables according to variational Bayesian models.

The generative adversarial network 76 uses two neural networks, a first of which generates candidates and the second of which evaluates the candidates. Typically, the generative network learns to map from a latent space to a particular data distribution of interest, taken from a training set, while the discriminative network discriminates between instances from the true data distribution and candidates produced by the generator. The generative network's training objective is to increase the error rate of the discriminative network by producing novel synthesized instances that appear to have come from the true data distribution. As the quality of the synthetic images at the generative network and the discrimination at the discriminative network increase, the features formed in the hidden layers of these networks become increasingly representative of the original data set, making them potentially useful features for defining the normal model.

The extracted features are then provided to the artificial neural network 64 which is trained on the extracted features to distinguish between the normal images and the test images. The validation images from the image database 62 can be provided to the artificial neural network 64, with a classifier evaluation component 66 calculating an accuracy of the artificial neural network on the validation images. In the illustrated implementation, this is performed after all training is completed, and the accuracy is the only performance metric calculated. The calculated accuracy is then provided to an anomaly detection component 68 that determines a likelihood of abnormalities in the tissue from the determined accuracy. In one implementation, the calculated likelihood can be a linear function of the accuracy, although it will be appreciated that non-linear or piecewise functions of the accuracy could also be utilized. The determined likelihood can be reported to a user via a user interface 69 at an associated display 54.

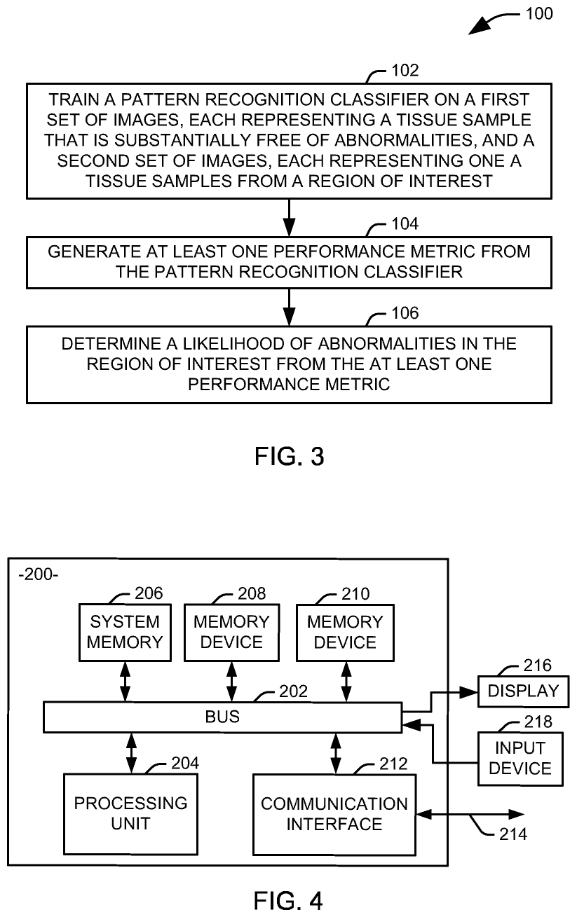

In view of the foregoing structural and functional features described above, a method in accordance with various aspects of the present invention will be better appreciated with reference to FIG. 3. While, for purposes of simplicity of explanation, the method of FIG. 3 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect the present invention.

FIG. 3 illustrates one example of a method 100 for screening a set of histopathology tissue samples representing a region of interest for abnormalities. At 102, pattern recognition classifier is trained on a first set of images, each representing a tissue sample that is substantially free of abnormalities, and a second set of images, each representing one of the set of histopathology tissue samples representing the region of interest. In one example, the anomaly detection system can represent the images as vectors of features, including features derived from color, texture, and gradient values extracted from the image as well as features derived from the latent space of an expert system applied to the image, such as a convolutional neural network, autoencoder, or generative adversarial network.

At 104, at least one performance metric from the pattern recognition classifier is generated. In this example, a given performance metric represents one of an accuracy of the classifier in discriminating between images representing tissue that is substantially free of abnormalities and images of histopathology tissue samples representing the region of interest and a training rate of the pattern recognition classifier, although it will be appreciated that other performance metrics could likely be utilized. At 106, a likelihood of abnormalities in the region of interest is determined from the at least one performance metric from the pattern recognition classifier. This likelihood can be provided to a user via an appropriate output device to support medical decision making on diagnosis of disorders within the region of interest or evaluating the effects of medication on the tissue in the region of interest.

In one implementation, the tissue sample is obtained from a patient (e.g., via a biopsy) and used to diagnose or monitor a medical condition in the patient. In another implementation, a therapeutic (e.g., a drug) can be administered to an animal subject for evaluation of the effects of the therapeutic on one or more organs of the subject. In yet another implementation, a therapeutic to a subject associated with the tissue sample after a first set of tissue samples has been evaluated. A second tissue sample can be extracted, analyzed, and compared to the first sample to determine an efficacy of the therapeutic in treating an existing condition.

FIG. 4 is a schematic block diagram illustrating an exemplary system 200 of hardware components capable of implementing examples of the systems and methods disclosed in FIGS. 1-3, such as the tissue screen system illustrated in FIGS. 1 and 2. The system 200 can include various systems and subsystems. The system 200 can be a personal computer, a laptop computer, a workstation, a computer system, an appliance, an application-specific integrated circuit (ASIC), a server, a server blade center, a server farm, etc.

The system 200 can includes a system bus 202, a processing unit 204, a system memory 206, memory devices 208 and 210, a communication interface 212 (e.g., a network interface), a communication link 214, a display 216 (e.g., a video screen), and an input device 218 (e.g., a keyboard and/or a mouse). The system bus 202 can be in communication with the processing unit 204 and the system memory 206. The additional memory devices 208 and 210, such as a hard disk drive, server, stand-alone database, or other non-volatile memory, can also be in communication with the system bus 202. The system bus 202 interconnects the processing unit 204, the memory devices 206-210, the communication interface 212, the display 216, and the input device 218. In some examples, the system bus 202 also interconnects an additional port (not shown), such as a universal serial bus (USB) port.

The processing unit 204 can be a computing device and can include an application-specific integrated circuit (ASIC). The processing unit 204 executes a set of instructions to implement the operations of examples disclosed herein. The processing unit can include a processing core.

The additional memory devices 206, 208 and 210 can store data, programs, instructions, database queries in text or compiled form, and any other information that can be needed to operate a computer. The memories 206, 208 and 210 can be implemented as computer-readable media (integrated or removable) such as a memory card, disk drive, compact disk (CD), or server accessible over a network. In certain examples, the memories 206, 208 and 210 can comprise text, images, video, and/or audio, portions of which can be available in formats comprehensible to human beings. Additionally or alternatively, the system 200 can access an external data source or query source through the communication interface 212, which can communicate with the system bus 202 and the communication link 214.

In operation, the system 200 can be used to implement one or more parts of a tissue screening system in accordance with the present invention. Computer executable logic for implementing the tissue screening system resides on one or more of the system memory 206, and the memory devices 208, 210 in accordance with certain examples. The processing unit 204 executes one or more computer executable instructions originating from the system memory 206 and the memory devices 208 and 210. The term “computer readable medium” as used herein refers to any medium that participates in providing instructions to the processing unit 204 for execution, and it will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof. In one example, the systems of FIGS. 1 and 2 can be implemented on one or more cloud servers and can be configured to receive feature sets for analysis from one or more client systems.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

From the above description of the invention, those skilled in the art will perceive improvements, changes, and modifications. Such improvements, changes, and modifications within the skill of the art are intended to be covered by the appended claims.

Having described the invention, We claim:

1. A system for screening a set of histopathology tissue samples representing a region of interest for abnormalities, comprising:
a processor; and
a non-transitory computer readable medium storing executable instructions comprising:
a pattern recognition classifier trained on a first set of images, each representing a tissue sample that is substantially free of abnormalities, and a second set of images, each representing one of the tissue samples representing the region of interest;
a classifier evaluation component that generates at least one performance metric from the pattern recognition classifier, a given performance metric representing one of an accuracy of the classifier in discriminating between images that are substantially free of abnormalities and images representing the set of tissue samples representing the region of interest and a training rate of the pattern recognition classifier;
an anomaly detection component that determines a likelihood of abnormalities in the region of interest from the at least one performance metric from the pattern recognition classifier; and
a user interface that provides the determined likelihood to a user at an associated output device.

2. The system of claim 1, further comprising a feature extractor which extracts a set of classification features from each of the first set of images and the second set of images.

3. The system of claim 2, wherein the set of classification features includes a set of features derived from a latent space of a variational autoencoder.

4. The system of claim 2, wherein the set of classification features includes a set of features derived from a hidden layer of a generative adversarial network.

5. The system of claim 2, wherein the set of classification features includes a set of features derived from a hidden layer of a convolutional neural network.

6. The system of claim 1, wherein the likelihood of abnormalities in the region of interest is determined as a function of the accuracy of the classifier in discriminating between images that are substantially free of abnormalities and images representing the set of tissue samples representing the region of interest.

7. The system of claim 1, wherein the likelihood of abnormalities in the region of interest is determined as a function of each of the accuracy of the classifier in discriminating between images that are substantially free of abnormalities and images representing the set of tissue samples representing the region of interest and the training rate of the classifier.

8. The system of claim 1, wherein the pattern recognition classifier comprises an artificial neural network.

9. The system of claim 8, wherein the pattern recognition classifier comprises an convolutional neural network.

10. A method for screening a set of histopathology tissue samples representing a region of interest for abnormalities, comprising:
training a pattern recognition classifier on a first set of images, each representing a tissue sample that is substantially free of abnormalities, and a second set of images, each representing one of the set of histopathology tissue samples representing the region of interest;
generating at least one performance metric from the pattern recognition classifier, a given performance metric representing one of an accuracy of the classifier in discriminating between images representing tissue that is substantially free of abnormalities and images of histopathology tissue samples representing the region of interest and a training rate of the pattern recognition classifier; and
determining a likelihood of abnormalities in the region of interest from the at least one performance metric from the pattern recognition classifier.

11. The method of claim 10, further comprising administering a therapeutic to a subject and extracting the histopathology tissue samples representing the region of interest from the subject.

12. The method of claim 10, further comprising extracting the histopathology tissue samples representing the region of interest via a biopsy of a human patient.

13. The method of claim 10, wherein determining a likelihood of abnormalities in the region of interest comprises determining the likelihood of abnormalities as a function of the training rate of the classifier.

14. The method of claim 13, wherein the function is a linear function.

15. The method of claim 10, further comprising extracting a plurality of features from the first set of images and the second set of images, the plurality of features including a set of features derived from one of a latent space of a variational autoencoder, a dense Speeded-Up Robust Features feature detection process, a set of multi-scale histograms of color and texture features, a set of latent vectors generated by a convolutional neural network, and a hidden layer of an generative adversarial network.

16. A system for screening a set of histopathology tissue samples representing a region of interest for abnormalities, comprising:
a processor; and
a non-transitory computer readable medium storing executable instructions comprising:
a pattern recognition classifier trained on a first set of images, each representing a tissue sample that is substantially free of abnormalities, and a second set of images, each representing one of the tissue samples representing the region of interest;
a classifier evaluation component that determines an accuracy of the classifier in discriminating between images that are substantially free of abnormalities and images representing the set of tissue samples representing the region of interest;
an anomaly detection component that determines a likelihood of abnormalities in the region of interest as a function of the determined accuracy of the classifier; and
a user interface that provides the determined likelihood to a user at an associated output device.

17. The system of claim 16, wherein an anomaly detection component that determines a likelihood of abnormalities in the region of interest as a linear function of the determined accuracy of the classifier.

18. The system of claim 16, wherein the pattern recognition classifier is a convolutional neural network.

19. The system of claim 16, wherein an anomaly detection component determines a likelihood of abnormalities in the region of interest as a function of the determined accuracy of the classifier and a training rate of the pattern recognition classifier.

20. The system of claim 16, further comprising a feature extractor which extracts a set of classification features from each of the first set of images and the second set of images, the set of classification features including a set of features derived from one of a latent space of a variational autoencoder, a dense Speeded-Up Robust Features feature detection process, a set of multi-scale histograms of color and texture features. a set of latent vectors generated by a convolutional neural network, and a hidden layer of an generative adversarial network.
